(19) United States
(12) Patent Application Publication
(10) Pub. No.: US 2025/0095800 A1
(43) Pub. Date: Mar. 20, 2025

(54) SYSTEMS AND METHODS FOR DESIGNING AND CONDUCTING CLINICAL TRIALS AND BIOMARKER VALIDATION STUDIES

(71) Applicant: Flagship Pioneering Innovations VII, LLC, Cambridge, MA (US)

(72) Inventors: Scott Logan Lipnick, Boston, MA (US); Katharine Maryell von Herrmann, Lexington, MA (US); Torben Lauesgaard Straight Nissen, Chestnut Hill, MA (US); William Zhenming Yuan, Somerville, MA (US); Xiaoyue Zhang, Revere, MA (US); Caleb John Kennedy, Cambridge, MA (US)

(21) Appl. No.: 18/889,904

(22) Filed: Sep. 19, 2024

Related U.S. Application Data
(60) Provisional application No. 63/584,033, filed on Sep. 20, 2023.

(51) Int. Cl. G16H 10/20

(52) U.S. Cl. CPC G16H 10/20

(57) ABSTRACT

In some embodiments, the subject matter of this disclosure relates to systems and methods for designing and conducting clinical trials and biomarker validation studies. An example method includes: obtaining access to a trained computer-implemented model; providing, to the trained computer-implemented model, a plurality of sets of values for a plurality of features, each set of values corresponding to a candidate individual from a set of candidate individuals; receiving, from the trained computer-implemented model, a prediction of a disease state for each set of values; identifying, from the predictions of the disease state, a group of participants from the set of candidate individuals; and facilitating at least one of a clinical trial or a biomarker validation study involving the group of participants.

### CROSS-REFERENCE TO RELATED APPLICATIONS

This application claims the benefit of U.S. Provisional Patent Application No. 63/584,033, filed Sep. 20, 2023, the entire contents of which are incorporated by reference herein.

### BACKGROUND

Clinical trials are essential for improving healthcare outcomes and advancing medical knowledge. A significant challenge in conducting clinical trials, however, is the recruitment of suitable participants. Recruiting the right participants is critical to ensuring the success and validity of a clinical trial. Despite its importance, current recruitment methods often face several challenges, including high costs, inefficiencies, and difficulties in finding and engaging eligible participants.

Likewise, biomarker validation studies are critical in the development of personalized medicine, therapeutic interventions, and diagnostic tools. Like clinical trials, however, a key challenge in conducting biomarker validation studies is the recruitment of suitable candidates. Biomarker validation can require participants who meet specific criteria, such as particular genetic profiles, disease stages, or pre-existing conditions.

There is a need for improved systems and methods for recruiting candidates for clinical trials, biomarker validation studies, or other longitudinal or cross-sectional investigations.

### SUMMARY

In various examples, this disclosure relates to systems and methods for designing and conducting clinical trials, biomarker validation studies, and other studies. A predictive model (e.g., a machine learning model) is trained to receive patient data as input and provide as output a prediction of a health condition or a disease state for the patient. The health condition or disease state can be or include, for example, a prediction along a health-to-disease axis or a disease risk axis and/or can be expressed as a probability distribution. Similar predictions can be made for other patients, and the predictions can be used to identify a group of participants for a clinical trial (or other study). For example, the clinical trial can have a target stage or target probability distribution for a disease of interest, and the model predictions can be used to select patients who satisfy the target stage or target probability distribution. In some instances, for example, patients can be selected such that predicted probability distributions for the patients add up to or approximate the target probability distribution for the clinical trial. Other patient characteristics, such as age, gender, or residential address, can also be considered when selecting the patients. The selected patients can then be included as participants in the clinical trial. Advantageously, in certain examples, the systems and methods described herein allow patients who satisfy requirements for clinical trials or other studies to be identified and selected more accurately and efficiently.

In one aspect, the subject matter of this disclosure relates to a method of performing a clinical trial or biomarker validation study. The method includes: obtaining access to a computer-implemented model that was trained using training data including a plurality of records for a plurality of individuals, each record including (i) values for a plurality of features for a respective individual from the plurality of individuals and (ii) a label providing an indication of a disease state for the respective individual; providing, to the trained computer-implemented model, a plurality of sets of values for the plurality of features, each set of values corresponding to a candidate individual from a set of candidate individuals; receiving, from the trained computer-implemented model, a prediction of a disease state for each set of values; identifying, from the predictions of the disease state, a group of participants from the set of candidate individuals; and facilitating at least one of a clinical trial or a biomarker validation study involving the group of participants.

In certain examples, the values for the plurality of features describe at least one of a demographic, a medical encounter, a physical examination, a diagnosis, a medication, a medical procedure, a vital measurement, a vaccination, a lab result, a serum, a urine sample, a bio-sample, a gene expression level, a medical image, a clinical note, a radiology report, a genetic test, a biomarker, a pathology report, health information, a social determinant of health, financial data, consumer data, or any combination thereof. The model can include at least one of a regression model, a classifier, a linear model, a non-linear model, a random forest, a kernel method, a Bayesian model, a decision tree, or a neural network. The prediction of the disease state can include a probability distribution. The prediction of the disease state can be on or associated with a primary stratification axis that is or includes a health-to-disease axis or a disease risk axis.

In some implementations, identifying the group of participants includes stratifying each candidate individual from the plurality of candidate individuals according to one or more primary stratification axes. Identifying the group of participants can further include stratifying each candidate individual from the plurality of candidate individuals according to one or more secondary stratification axes. The one or more secondary stratification axes can represent one or more properties of the plurality of individuals, and the properties can include at least one of a physiological comorbidity, a demographic, or a socioeconomic variable. At least one stratification axis from the one or more primary stratification axes or the one or more secondary stratification axes can represent at least one of a continuous dimension or an ordinal dimension. A position along at least one stratification axis from the one or more primary stratification axes or the one or more secondary stratification axes can be represented by a point, a point estimate, or a probability distribution.

In various instances, the clinical trial or the biomarker validation study can include studying at least one of a progression of transitions in health, validity of a biomarker, validity of a panel of biomarkers, validity of a target therapeutic, efficacy of a therapeutic intervention, efficacy of a digital intervention, efficacy of a behavioral intervention, or any combination thereof. The group of participants can be enriched in one or more properties relative to the set of candidate individuals, the one or more properties including reduced heterogeneity in physiology, increased probability of outcome events, increased propensity to respond to an intervention, increased likelihood of observing specific physiological or pathological signs, or any combination thereof.

In some examples, identifying the group of participants includes: determining a target distribution for the set of candidate individuals according to a goal of the clinical trial or the biomarker validation study, the target distribution defining a probability distribution; and selecting the group of participants from the set of candidate individuals to achieve a collective distribution for the group of participants that resembles the target distribution. The target distribution can be determined based on at least one parameter to be evaluated in the clinical trial or the biomarker validation study, the at least one parameter including at least one of a biological property, a biomarker, a panel of biomarkers, a therapeutic target, or a pharmacologic intervention. The target distribution can be constant or uniform with respect to the disease state (e.g., along a health-to-disease axis). The target distribution can include a risk probability distribution. The target distribution can be enriched with respect to a region of a stratification axis associated with the disease state. Selecting the group of participants can include minimizing a number of individuals in the group of participants. Selecting the group of participants can include: identifying multiple groups of individuals, wherein each group of individuals from the multiple groups of individuals has a similar probability distribution with respect to the disease state; and determining a number of individuals from each group of individuals (and/or individuals not in any group) to include in the group of participants to satisfy the target distribution.

In various instances, the method includes facilitating the clinical trial, and the clinical trial includes evaluating the efficacy of a treatment. Facilitating the clinical trial can include registering members from the group of participants to the clinical trial, such that the members are administered a medication, a digital intervention, or a behavioral intervention. The method can include facilitating the biomarker validation study, and the biomarker validation study can include validating a biomarker or panel of biomarkers.

In various implementations, identifying the group of participants includes achieving a desired distribution of at least one covariate, the at least one covariate including a demographic, a comorbidity, a medication usage, a medical history, a socioeconomic property, a biological property, or any combination thereof. The method can include training the computer-implemented model using the training data.

In another aspect, the subject matter of this disclosure relates to a system for performing a clinical trial or biomarker validation study. The system includes one or more computer processors programmed to perform operations including: accessing or obtaining access to a computer-implemented model that was trained using training data including a plurality of records for a plurality of individuals, each record including (i) values for a plurality of features for a respective individual from the plurality of individuals and (ii) a label providing an indication of a disease state for the respective individual; providing, to the trained computer-implemented model, a plurality of sets of values for the plurality of features, each set of values corresponding to a candidate individual from a set of candidate individuals; receiving, from the trained computer-implemented model, a prediction of a disease state for each set of values; identifying, from the predictions of the disease state, a group of participants from the set of candidate individuals; and facilitating at least one of a clinical trial or a biomarker validation study involving the group of participants.

In certain examples, the prediction of the disease state can be on or associated with a primary stratification axis including a health-to-disease axis or a disease risk axis. Identifying the group of participants can include stratifying each candidate individual from the plurality of candidate individuals according to one or more primary stratification axes and/or one or more secondary stratification axes.

Identifying the group of participants can include: determining a target distribution for the set of candidate individuals according to a goal of the clinical trial or the biomarker validation study, the target distribution defining a probability distribution; and selecting the group of participants from the set of candidate individuals to achieve a collective distribution for the group of participants that resembles or approximates the target distribution. The target distribution can include a risk probability distribution. Selecting the group of participants can include: identifying multiple groups of individuals, wherein each group of individuals from the multiple groups of individuals has a similar probability distribution with respect to the disease state; and determining a number of individuals from each group of individuals (and/or individuals not in any group) to include in the group of participants to satisfy the target distribution. The operations can include training the computer-implemented model using the training data.

These and other objects, along with advantages and features of embodiments of the present invention herein disclosed, will become more apparent through reference to the following description, the figures, and the claims. Furthermore, it is to be understood that the features of the various embodiments described herein are not mutually exclusive and can exist in various combinations and permutations.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A and 1B are plots of number of individuals versus disease state along a health-to-disease axis for a given disease, in accordance with certain embodiments.

FIG. 2 is a schematic diagram of a method of identifying a cohort of individuals for a clinical trial or other study, in accordance with certain embodiments.

FIG. 3 is a schematic diagram of a method of training a machine learning model, in accordance with certain embodiments.

FIG. 4 is a schematic diagram of a method of using a predictive model to identify a cohort of individuals for participation in a study, in accordance with certain embodiments.

FIG. 5 is a block diagram of an example computer system, in accordance with certain embodiments.

### DETAILED DESCRIPTION

A description of example embodiments follows. It is contemplated that apparatus, systems, methods, and processes of the claimed invention encompass variations and adaptations developed using information from the embodiments described herein. Adaptation and/or modification of the apparatus, systems, methods, and processes described herein may be performed by those of ordinary skill in the relevant art.

It should be understood that the order of steps or order for performing certain actions is immaterial so long as the invention remains operable. Moreover, two or more steps or actions may be conducted simultaneously.

In various examples, an “organism” (alternatively referred to herein as an “individual,” a “subject,” or a “patient”) can refer to an animal, a plant, a fungus, or any living thing. The organism can be, for example, a mammal, such as a human or a mouse.

In various examples, a “disease” can refer to any disorder of function or structure in an organism. The disease can be any type of disease, including, for example, cancer (e.g., colorectal cancer, lung cancer, skin cancer, etc.), genetic diseases (e.g., spinal muscular atrophy (SMA), Huntington's disease (HD), or Duchenne's muscular dystrophy (DMD)), liver disease (e.g., metabolic dysfunction-associated steatotic liver disease (MASLD), metabolic dysfunction-associated steatohepatitis (MASH), or cirrhosis), kidney disease (e.g., chronic kidney disease (CKD) or polycystic kidney disease), diabetes (e.g., Type II diabetes), neurodegenerative disorders (e.g., Alzheimer's disease (AD), amyotrophic lateral sclerosis (ALS), or Parkinson's disease (PD)), autoimmune disorders (e.g., multiple sclerosis (MS), rheumatoid arthritis (RA), or inflammatory bowel disorders (IBD)), pulmonary diseases (e.g., chronic obstructive pulmonary disease (COPD) or pulmonary fibrosis (e.g., IPF)), long COVID, colon polyps, age-related macular degeneration (AMD), or any other type of disease. In some examples, the disease can be, include, and/or exhibit inflammation, cellular reaction to stress, or other types of dysfunction.

In various examples, a “disease of interest” can refer to a disease that is being characterized or analyzed using the predictive modeling techniques described herein. For example, a machine learning model can be trained to make predictions related to the disease of interest.

In various examples, a “reference domain” (alternatively referred to herein as a “feature space”) can be or include a collection of features or variables (e.g., biological data, latent variables, or phenotype specific variables) that can be used as training data for training a machine learning model and/or as inputs to the machine learning model. The reference domain can include, for example, gene expression levels and/or other biological features related to a disease of interest.

In various examples, a “biomarker” can refer to a measurable indicator of a biological condition or state. A biomarker can signal the presence or progression of a disease or the efficacy of a treatment. A biomarker can be a biological molecule and can be found in tissues, blood, or bodily fluids. A biomarker can be used for diagnostic purposes, for example, to confirm or detect presence of a condition or disease of interest or to identify individuals having a subtype of the disease. A biomarker can be measured repeatedly to monitor individuals, for example, to assess status of a medical condition or a disease or to look for evidence of an effect of (or exposure to) an environmental agent or a medical product.

In certain examples, one or more predictive models (e.g., machine learning models or computer-implemented models) are used to identify individuals for inclusion in a clinical trial, a biomarker validation study, a cross-sectional study, or longitudinal study (each referred to herein as “trial” or “study” for simplicity). The one or more predictive models can be configured to receive health data for an individual as input and provide a predicted disease state, health condition, and/or disease risk for the individual as output. Such predictions can be made for a set of individuals. Based on the predictions, a cohort of individuals can be selected that satisfies a target disease state, target health condition, and/or target disease risk for the clinical trial or other study.

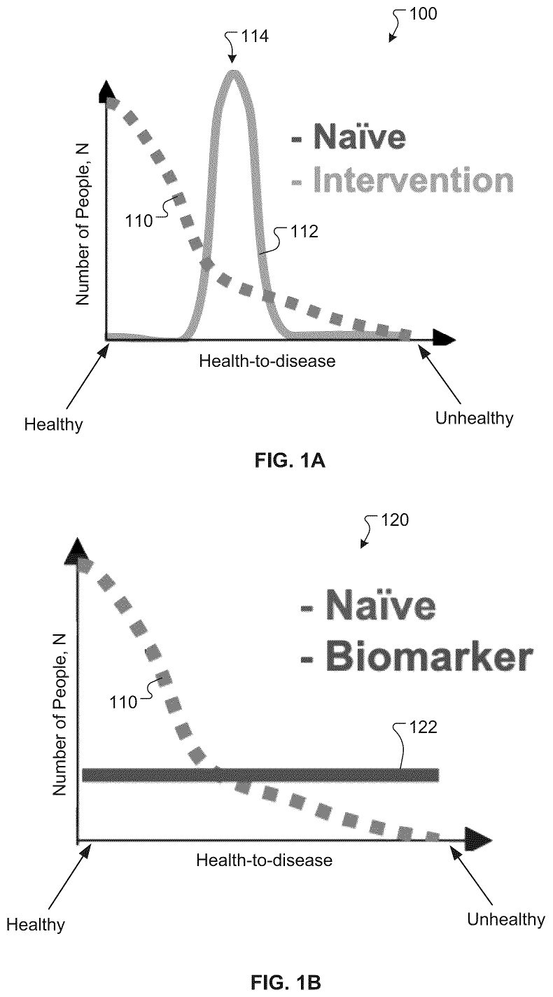

FIG. 1A is a plot 100 of number of individuals N versus disease state along a health-to-disease axis for a given disease, in accordance with certain examples. The plot 100 includes population distributions for two cohorts. A first population distribution 110 is for a cohort that is naively selected from a general population and illustrates that most individuals are healthy. A second population distribution 112 is for a cohort corresponding to a target stage 114 of disease for a clinical trial. For example, the cohort in the second population distribution 112 may be of interest for prescribing and evaluating a treatment in the clinical trial. In various examples, the cohort for the second population distribution 112 can be identified using the systems and methods described herein. The target stage 114 can correspond to a range or value for disease severity or risk. For example, the target stage can be correspond to a numerical range or value for eGFR in chronic kidney disease, visual acuity in age-related macular degeneration, time-to-diagnosis in Parkinson's disease, or diabetes risk (e.g., risk of developing diabetes).

FIG. 1B is a similar plot 120 of number of individuals N versus disease state along the health-to-disease axis for a given disease, in accordance with certain examples. The plot 120 includes the first population distribution 110 corresponding to the naively selected cohort. The plot 120 also includes a third population distribution 122 having an even or uniform representation across the health-to-disease axis. In various examples, the third population distribution 122 is desirable for biomarker validation studies in which biomarkers can be assessed for reliability and accuracy.

In certain examples, the cohorts from the second population distribution 112 and the third population distributions 122 can be identified using the systems and methods described herein. For example, a machine learning model (or other predictive model) can be configured to receive health information for individuals as input and determine a disease state or health condition for the individuals as output. To create the cohort for the second population distribution 112, the machine learning model can be used to identify individuals who have a disease state corresponding to the target stage 114 targeted for the clinical trial. Such individuals can be included in the cohort while other individuals having a disease state that falls outside of the target stage 114 can be excluded from the cohort.

Likewise, to create the cohort for the third population distribution 122, the machine learning model can be used to identify and select individuals who have a variety of disease states, ranging from healthy to unhealthy across the health-to-disease axis. In some examples, the uniform representation of the third population distribution 122 can be obtained by selecting a combination of individuals who are healthy (e.g., low risk of developing the disease), moderately healthy (e.g., medium risk of developing the disease), unhealthy (e.g., high risk of developing the disease), and/or at other, intermediate disease stages. The combined disease states of the selected individuals can achieve or approximate the uniform representation of the third population distribution 122.

In various examples, the selection of individuals for a cohort can be made by considering a risk probability distribution for each individual. For example, each individual can have a risk probability distribution along the health-to-disease axis, and the cohort can be selected such that the risk probability distributions for the individuals in the cohort add up to a desired population distribution (or an approximation thereof), such as the third population distribution 122. In some examples, a risk probability distribution provides probabilities associated with an individual's risk of having or developing a disease. Additionally or alternatively, a risk probability distribution can be correlated with or derived from a predicted health condition for an individual along the health-to-disease axis. For example, an individual who is predicted to be healthy can have a risk probability distribution indicating the individual has a low risk of having or developing the disease. Likewise, an individual who is predicted to be unhealthy can have a risk probability distribution indicating the individual has a high risk of having or developing the disease. There can be multiple risk levels for a disease, such as low, mid-low, mid-high, or high, and the risk probability distribution can provide a probability value for each risk level. Risk probabilities and risk probability distributions can be calculated using the predictive models described herein.

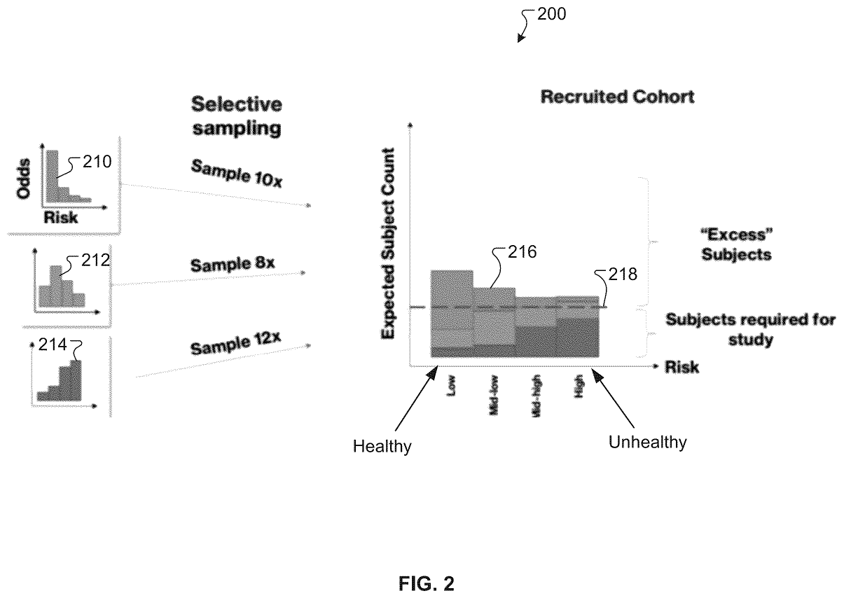

FIG. 2 is a schematic diagram of a method 200 of identifying a desired cohort, in accordance with certain examples. Risk probability distributions 210, 212, and 214 are calculated for individual patients using a predictive model. In the depicted example, patients fall into three broad groups: a low risk group (with risk distributions shifted lower) represented by the risk probability distribution 210, a medium risk group (with risk distributions peaking in a center region) represented by the risk probability distribution 212, and a high risk group (with risk distributions shifted higher) represented by the risk probability distribution 214. A number of patients from each group can be selected or sampled to assemble a final cohort. In the depicted example, 10 patients are sampled from the low risk group, 8 patients are sampled from the medium risk group, and 12 patients are sampled from the high risk group. Additionally or alternatively, one or more patients who have unique risk probability distributions (e.g., different from risk probability distributions 210, 212, and 214) can be selected, if desired. The resulting cohort in the depicted example is composed of low, moderate, and high risk subjects. A histogram 216 for the cohort shows an estimated number of patients in each of the following risk categories: low, mid-low, mid-high, and high. A threshold line 218 corresponds to a target distribution for a study involving the cohort, for example, as specified by a sponsor of the study.

In some examples, “excess” subjects can be recruited to ensure that an overall density or number of patients is above the threshold line 218 for all risk categories. For example, a minimum number of patients can be selected that satisfies or approximates the target distribution and has a threshold number of patients within each risk category. Advantageously, the approach allows a recruited cohort meeting target parameters to be selected from risk probability distributions, even if the model is unable to precisely place any individual subject along the health-to-disease axis. While the target distribution in this example (represented by the threshold line 218) is constant or uniform, it is understood that the target distribution can be nonuniform. For example, a target distribution can resemble risk probability distribution 210, 212, or 214.

In some embodiments involving biomarker validation, it can be desirable to obtain an even distribution of individuals along the health-to-disease axis, disease risk axis, or other dimension (e.g., represented by the third population distribution 122 or the threshold line 218). Advantageously, the method 200 can utilize predictive modeling and probability distribution-based sampling to achieve the desired distribution. In certain examples, one or more models (referred to herein as “model” for simplicity) can generate a probability distribution representing a health condition or risk probability for each individual, and the model can aggregate individuals to make broader population distributions of interest. The approach can provide an expected subject count or a projected size for a recruited cohort. The subject count can be used to evaluate the quality of the method 200 or other method used to identify a cohort. In general, a stronger method is able to satisfy trial requirements using a smaller subject count.

In some embodiments, one or more stratification axes can be selected for a group of individuals (referred to as “candidate individuals”) who are being considered for a study, and the model can stratify each individual in the group according to the one or more stratification axes. A stratification axis can define, relate to, and/or provide a measure of one or more inclusion criteria for the study. Such criteria can include one or more primary stratification axes and optionally one or more secondary stratification axes. The one or more primary stratification axes can relate to or provide a measure of health or disease, such as, for example, a disease state or stage (e.g., along the health-to-disease axis), a health condition, and/or a disease risk (e.g., a risk of having or developing a disease, along a disease risk axis). The one or more secondary stratification axes can include or relate to other characteristics, such as, for example, gender, age, ethnicity, a demographic, treatment history, medication usage, a physiological comorbidity, a socioeconomic variable, or any combination thereof. For instance, in addition to recruiting evenly across a health-to-disease axis (e.g., a primary stratification axis), a designer of a trial (e.g., a trial sponsor) might want an equal number of male and female participants (e.g., a secondary stratification axis). In a specific example involving chronic kidney disease, for example, it may be desirable to select participants who have a certain distribution of eGFR values (e.g., on a primary stratification axis for eGFR) and a certain distribution across age, sex, and/or distance to the clinical site (three secondary stratification axes).

In various examples, the model and/or related techniques can be used to identify patients to be recruited for the study as a function of their position or probability distribution along the selected axes. The model can be used to select and/or identify a subpopulation of the candidate individuals whose collective distribution of positions or probabilities along one or more stratification axes is projected to resemble or approximate a target distribution (e.g., the second or third population distribution 112 or 122) for the trial. Such predictions can be used to recruit the selected individuals for the study, for example, via automatic generation of invitation letters, emails, or notifications to health care providers through an electronic health care records system. Alternatively or additionally, selected individuals can be recruited via primary care providers, specialty care providers, or other settings. In some examples, information related to the stratification axes can be obtained from a database of records for the candidate individuals.

Additionally or alternatively, risk probabilities or risk probability distributions can used to make treatment decisions for patients. For example, data for a patient can be provided to the predictive models described herein, and the predictive models can determine and/or output a probability distribution representing a health of the patient (e.g., a risk probability distribution). The probability distribution can be used by the patient, a doctor, and/or other healthcare professionals to make healthcare and/or treatment decisions for the patient. In some examples, the predictive models can use the probability distribution to determine and/or output one or more treatment recommendations for the patient. In some examples, the probability distributions can be used to derive metrics such as disease risk or percent chance that are more familiar and interpretable to patients or doctors.

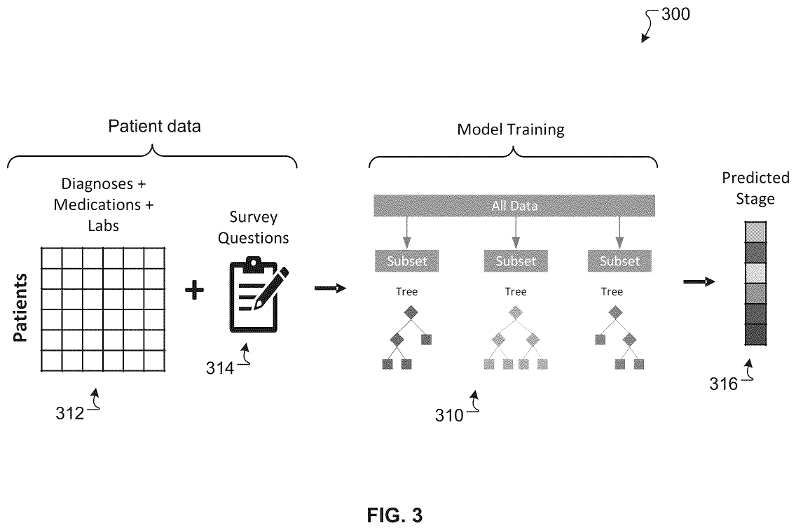

FIG. 3 is a schematic diagram of a method 300 of training a machine learning model 310, in accordance with certain examples. In some embodiments, the model 310 can be or include a regression model, a classifier, a linear model, a non-linear model, a random forest, a kernel method, a Bayesian model, a decision tree (e.g., XGBoost), a neural network, a generative model, or other type of machine learning model. In certain examples, the model 310 can be or include, for example, a multi-layer fully connected neural network.

In various examples, the model 310 can be trained to receive data for an individual as input and to provide a predicted disease state, health condition, and/or disease risk for the individual as output. The input data used to train the model 310 can include, for example, patient data 312 (e.g., medications, diagnoses, labs, vitals, etc.) and/or survey data 314 (if available). The model inputs can form a reference domain or feature space for the model 310. In some embodiments, the patient data 312 can include real-world data that exists in medical records or can be created with labs, imaging, other tests, consumer data, social determinants of health, genetic data, or any other relevant source. Such data can be directly measured or imputed (e.g., using a regression model). The survey data 314 can be derived from question(s) and corresponding answer(s) and/or can represent additional data captured in a prospective manner for each subject that may not be readily accessible from medical records or other data sources. The survey data 314 and/or the patient data 312 can include, for example, waist-to-hip ratio, waist circumference, hip circumference, body fat percentage, blood pressure, employment status, diet, exercise, etc. In some instances, the model input data can include information related to demographics (e.g., gender, ethnicity, income, etc.).

In certain examples, the training data and/or model outputs can include a continuous representation of health 316, which can be or include a predicted value or score along a continuous dimension of health (e.g., the health-to-disease axis). In certain examples, the continuous representation of health 316 can underly current staging paradigms, which are typically abstracted to high-level categories such as “stage II” or “stage IV”. The continuous representation of health 316 can serve as labels or ground truth data in the training data. The continuous representation of health 316 can be or include, for example, image-based fat fraction for nonalcoholic fatty liver disease (NAFLD), estimated Glomerular Filtration Rate (eGFR) for Chronic Kidney Disease (CKD), and/or Unified Parkinson's Disease Rating Scale (UPDRS) for Parkinson's disease (PD).

Other features can be used as model inputs and/or model outputs (e.g., in the training data). For example, the model inputs and/or model outputs can include propensity for a transition, time to a transition, biological stage, physiological stage, and/or any measured or forecasted dimension or variable aligned with transitions from health to disease. For PD, for example, model inputs can include data related to brain imaging, functional assessments by specialists, a diagnostic trajectory, a social determinant of health, or any combination thereof. For diabetes, for example, model inputs can include data related to dietary recall, lifestyle factors, medical history, blood lab levels, or any combination thereof. Model outputs can include an estimated time to diagnosis (e.g., for PD) and/or a risk of developing a disease (e.g., diabetes). The risk of developing a disease can be stratified into high, medium, and low-risk cohorts.

With enough ground truth data, the model can be trained to analyze individuals who have or do not have a disease or condition. This enables interpretability and/or transferability of learned information across healthcare systems and/or time, and it allows the model to make predictions for data naïve individuals (e.g., people yet to be analyzed by the system or who were not used as training data sources).

In some examples, the model 310 can be trained to selectively enrich for desired populations. This can involve isolating a cohort of individuals across a relevant dimension (e.g., health condition), establishing cohort definitions with precision at or near specific points along the dimension, and/or identifying patterns in features that recognize the cohort. The cohort can be selected for inclusion in a clinical trial or other study, as described herein.

In some cases, data used to generate training data or model inputs (e.g., lab results, imaging results, and/or other patient data) can have a lack of cohesion in formatting, language, and/or terminology. Mapping such data to a uniform or consistent format, language, and/or terminology can be beneficial for training and using the model.

In various examples, the model can be used to determine when different therapeutic targets or interventions are relevant and/or effective. For example, some intervention inhibiting lipid accumulation can be delivered earliest in NAFLD, whereas an intervention inhibiting a factor recruiting immune cells or activating fibrotic cells can be applied at a later stage of progression. One or more predictive biomarkers can be used (e.g., by the model) to identify individuals who are at a certain stage of disease and/or who are more likely than other individuals to experience a favorable or unfavorable effect from the therapeutic targets or interventions.

In some embodiments, a continuous time scale can be generated and employed using the systems and methods described herein. In some embodiments, the model can be trained using labels from subpopulations to provide labels for a new or different subpopulation. Training data can be generated by extracting PDFF (e.g., fat fraction) from individuals with MRI data and/or by generating eGFR from individuals with standard lab tests. These generated or extracted labels or values can be used to train the model to learn how to predict these features in other individuals based on more traditional data (e.g., derived from electronic health records and/or surveys) that are easy to acquire or passively collected.

In some embodiments, two approaches can be used to identify and validate biomarkers. In a first approach, biological studies are employed in which snRNA-seq data is generated from tissues collected across a spectrum of health to disease (e.g., multiple molecular stages), and then proteins that are secreted and likely to be detected in the blood are identified. In a second approach, blood is collected from individuals who are recruited at various stages of the health to disease continuum. Studies can then be performed on the blood to identify or determine changes with stages of progression. The studies can be biased, unbiased, proteomic, metabolomic, lipidomic, any combination of the above, etc.

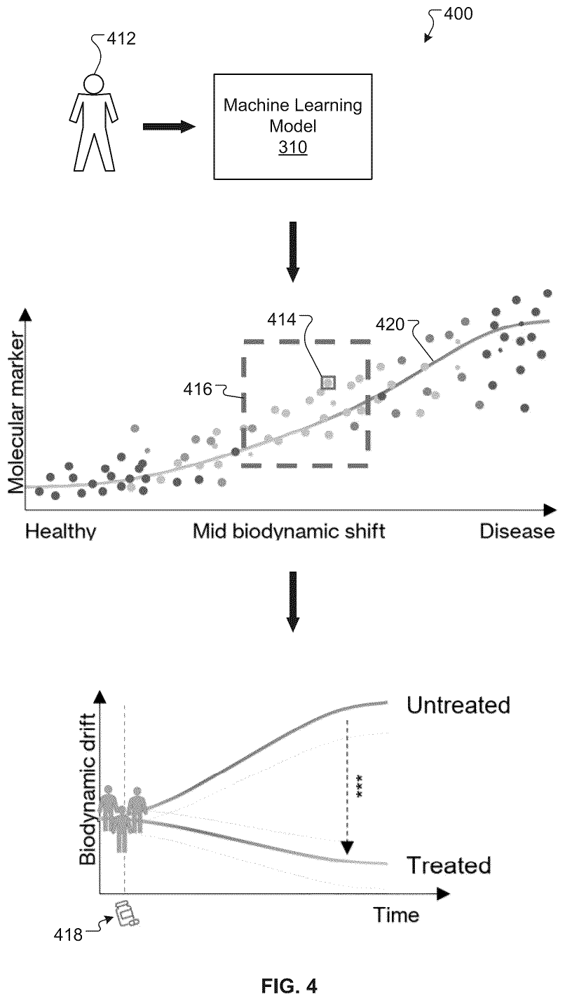

FIG. 4 is a schematic diagram of a method 400 of using a predictive model (e.g., the model 310) to identify a cohort of individuals for participation in a clinical trial or study, in accordance with certain examples. Health data, demographic data, and/or survey data (e.g., the patient data 312 and the survey data 314) for an individual 412 are provided as input to the predictive model 310. The data can be entered via a user interface by the individual 412, a doctor, a healthcare professional, a clinical trial designer, or other person, or the data can be entered via an automated data pull via an electronic health record (EHR) system. The model 310 can provide as output a prediction of a health condition (e.g., the continuous representation of health 316), disease state, and/or disease risk for the individual. For example, the model 310 can forecast a value or condition for a molecular marker (e.g., a gene expression level) across a spectrum from health to disease for the individual 412. Similar predictions can be made for other individuals. In some examples, the spectrum from health to disease can capture temporality of a dysfunction without having to do longitudinal tracking of individuals.

In the depicted example, a condition 414 for the individual 412 is within a target state 416 that lies between a healthy state and a diseased state. The target state 416 can correspond to an intended target population for the clinical trial. For example, the target state 416 can be selected to capture individuals who are most likely to respond to a treatment associated with the clinical trial. In many chronic diseases, such as diabetes, IBD, or age-related macular degeneration (AMD), different therapies can be used for patients depending on disease severity (e.g., early, moderate, or severe disease). With the target state 416 identified, the model predictions are used to identify a cohort of individuals who have health conditions (e.g., the condition 414) falling within the target state 416. Alternatively or additionally, the cohort can be identified using probability distributions (e.g., risk probability distributions 210, 212, and/or 214) that satisfy the target state 416 or approximate a target distribution, as described herein. Such individuals can be recruited for the clinical trial, as described herein. Once the individuals have been recruited, the clinical trial can be run by providing an intervention, therapy, or treatment 418 to some or all of the recruited individuals. For example, the treatment 418 can be provided to a first portion of the individuals in a test group, and a placebo can be provided to a second portion of the individuals in a control or placebo group. Results from the trial can be used to determine whether the treatment has a positive outcome on the individuals who received the treatment.

In certain examples, the model 310 can provide a prediction of disease risk for the individual 412 and other candidate individuals considered for the clinical trial. The disease risk can correspond to a transition from a healthy state to a disease state. The transition can include, for example, an early biodynamic shift, a mid-biodynamic shift, or a late biodynamic shift. Such biodynamic shifts can be cascades with different characteristics at early, mid, and late stages of disease. The cohort of individuals identified for the study can be used to determine a relationship 420 between levels of a molecular marker and a biodynamic shift between health and disease. The relationship 420 can define a trend over all sampled individuals. In the depicted example, the target state 416 corresponds to a mid-biodynamic shift. Because such individuals may be more likely to progress towards disease than healthy individuals, it can be easier to observe a treatment effect during the trial, if one exists.

In embodiments, systems and methods described herein, including the predictive models, can be implemented over a network (e.g., including the Internet). For example, the predictive models can be accessed by a clinical study designer over the network, where the predictive models identify or provide a cohort of individuals over a HIPAA compliant and secure connection. Further, the cohort can be provided in a double-blind format such that participants can be contacted for enrollment in the study while their identities are shielded from scientists running the study.

In various examples, the systems and methods described herein can be used to facilitate a clinical trial, a biomarker validation study, or other study. Facilitating the clinical trial (or other study) can include a variety of actions related to organizing or conducting the trial. Such actions can include, for example: recruiting a group of participants (e.g., identified using the systems and methods described herein) for the trial or study; including or registering a group of participants in the trial or study; collecting biospecimens, imaging data, or other data as part of the trial or study; treating one or more trial or study participants with an intervention; applying a diagnostic to one or more participants; making a recommendation to one or more participants about treatment decisions; making a recommendation to one or more participants about diagnostic or screening tests; or any combination thereof.

### Machine Learning and Neural Networks

Machine learning is a method of teaching computers to learn and make decisions on their own, without explicitly being programmed to perform a specific task. It involves feeding a large amount of data into a computer program, which then uses statistical analysis to identify patterns and relationships within the data. The goal is to enable the program to make predictions or decisions based on these patterns and relationships, without being explicitly told how to do so.

Neural networks are a type of machine learning algorithm that are inspired by the structure and function of the human brain. They consist of layers of interconnected “neurons,” sometimes called nodes, which process and transmit information. Each neuron receives input from other neurons, processes it, and passes it on to other neurons in the next layer.

The layers in a neural network refer to the layers of interconnected neurons. There are typically multiple layers in a neural network, with the input layer receiving the raw data and the output layer producing the final prediction or decision. Between the input and output layers, there are one or more hidden layers, which process the data and pass it on to the next layer.

By training a neural network on a large dataset, the connections between neurons (called “weights”) can be adjusted to improve the network's ability to make predictions or decisions. To train a neural network, the data is fed through the network and the output is compared to the desired result. If the output is not accurate, the weights are adjusted to reduce the error. This process is repeated multiple times, with the network continually adjusting the weights to improve its accuracy. Once the network has been trained, it can be used to make predictions or decisions on new data, based on the patterns and relationships it has learned from the training data.

In various examples, “machine learning” can refer to the application of certain techniques (e.g., pattern recognition and/or statistical inference techniques) by computer systems to perform specific tasks. Machine learning techniques (automated or otherwise) may be used to build data analytics models based on sample data (e.g., “training data”) and to validate the models using validation data (e.g., “testing data”). The sample and validation data may be organized as sets of records (e.g., “observations” or “data samples”), with each record indicating values of specified data fields (e.g., “independent variables,” “inputs,” “features,” or “predictors”) and corresponding values of other data fields (e.g., “dependent variables,” “outputs,” or “targets”). Machine learning techniques may be used to train models to infer the values of the outputs based on the values of the inputs. When presented with other data (e.g., “inference data”) similar or related to the sample data, such models may accurately infer the unknown values of the targets of the inference data set. Such models can be referred to herein as “machine learning models,” predictive models,” or “computer-implemented models.”

A feature of a data sample may be a measurable property of an entity (e.g., a cell, a biological sample, a person, thing, event, activity, etc.) represented by or associated with the data sample. For example, a feature can be a characteristic of a cell of an organism. As a further example, a feature can be a gene expression level associated with the cell. In some cases, a feature of a data sample is a description of (or other information regarding) an entity represented by or associated with the data sample. A value of a feature may be a measurement of the corresponding property of an entity or an instance of information regarding an entity. For instance, in the above example in which a feature of a cell is the gene expression level, a value of the ‘expression of gene G’ feature can be 215,000, which is the number of messenger RNA fragments in that cell that map to the region of gene G on the human reference genome. In some cases, a value of a feature can indicate a missing value (e.g., no value). For instance, in the above example in which a feature is the gene expression level, the value of the feature may be ‘NULL,’ indicating that the gene level was not measured by a given technology.

Features can also have data types. For instance, a feature can have an image data type, a numerical data type, a text data type (e.g., a structured text data type or an unstructured (“free”) text data type), a categorical data type, or any other suitable data type. In the above example, the feature of a shape extracted from an image of a cell can be of an image data type. In general, a feature's data type is categorical if the set of values that can be assigned to the feature is finite.

As used herein, the “development” of a machine learning model may refer to construction of the machine learning model. Machine learning models may be constructed by computers using training data sets. Thus, “development” of a machine learning model may include the training of the machine learning model using a training data set. In some cases (generally referred to as “supervised learning”), a training data set used to train a machine learning model can include known outcomes (e.g., labels or target values) for individual data samples in the training data set. For example, when training a supervised computer vision model to detect images of cats, a target value for a data sample in the training data set may indicate whether the data sample includes an image of a cat. In other cases (generally referred to as “unsupervised learning”), a training data set does not include known outcomes for individual data samples in the training data set.

Following development, a machine learning model may be used to generate inferences with respect to “inference” data sets. For example, following development, a computer vision model may be configured to distinguish data samples including images of cats from data samples that do not include images of cats. As used herein, the “deployment” of a machine learning model may refer to the use of a developed machine learning model to generate inferences about data other than the training data.

### Computer Implementations

In some examples, some or all of the processing described above can be carried out on a personal computing device, on one or more centralized computing devices, or via cloud-based processing by one or more servers. Some types of processing can occur on one device and other types of processing can occur on another device. Some or all of the data described above can be stored on a personal computing device, in data storage hosted on one or more centralized computing devices, and/or via cloud-based storage. Some data can be stored in one location and other data can be stored in another location. In some examples, quantum computing can be used and/or functional programming languages can be used. Electrical memory, such as flash-based memory, can be used.

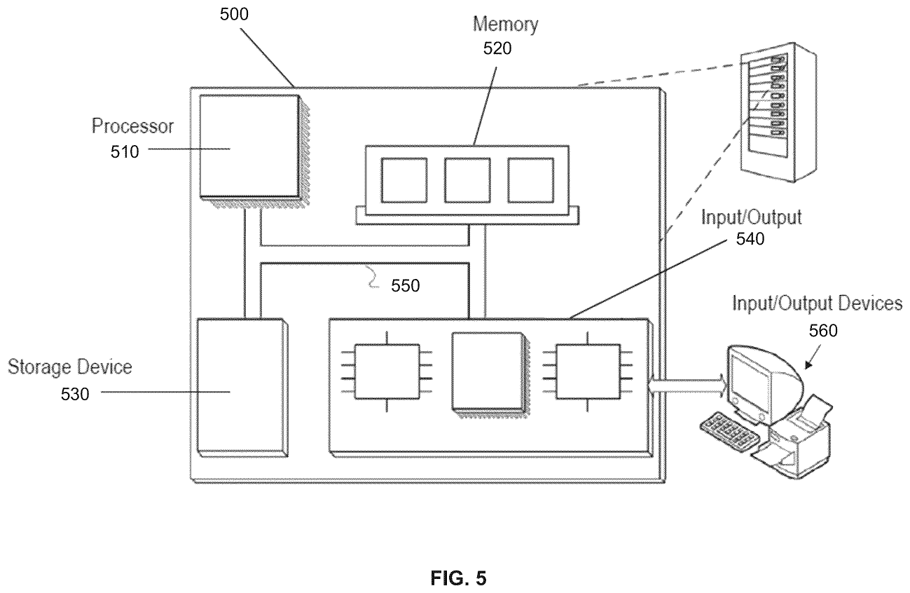

FIG. 5 is a block diagram of an example computer system 500 that may be used in implementing the technology described in this document. General-purpose computers, network appliances, mobile devices, or other electronic systems may also include at least portions of the system 500. The system 500 includes a processor 510, a memory 520, a storage device 530, and an input/output device 540. Each of the components 510, 520, 530, and 540 may be interconnected, for example, using a system bus 550. The processor 510 is capable of processing instructions for execution within the system 500. In some implementations, the processor 510 is a single-threaded processor. In some implementations, the processor 510 is a multi-threaded processor. The processor 510 is capable of processing instructions stored in the memory 520 or on the storage device 530.

The memory 520 stores information within the system 500. In some implementations, the memory 520 is a non-transitory computer-readable medium. In some implementations, the memory 520 is a volatile memory unit. In some implementations, the memory 520 is a nonvolatile memory unit.

The storage device 530 is capable of providing mass storage for the system 500. In some implementations, the storage device 530 is a non-transitory computer-readable medium. In various different implementations, the storage device 530 may include, for example, a hard disk device, an optical disk device, a solid-date drive, a flash drive, or some other large capacity storage device. For example, the storage device may store long-term data (e.g., database data, file system data, etc.). The input/output device 540 provides input/output operations for the system 500. In some implementations, the input/output device 540 may include one or more of a network interface devices, e.g., an Ethernet card, a serial communication device, e.g., an RS-232 port, and/or a wireless interface device, e.g., an 802.11 card, a wireless modem (e.g., 3G, 4G, or 5G). In some implementations, the input/output device may include driver devices configured to receive input data and send output data to other input/output devices, e.g., keyboard, printer and display devices 560. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 530 may be implemented in a distributed way over a network, for example as a server farm or a set of widely distributed servers, or may be implemented in a single computing device.

Although an example processing system has been described in FIG. 5, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, an engine, a pipeline, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### Some Embodiments

In some embodiments, a method of running a cross-sectional or longitudinal human study or trial includes separating data from a database of records of a plurality of individuals into input data and output data, wherein the input data is static or temporal features of a respective individual collected either passively or through active engagement, and wherein the output data is a primary stratification axis that represents transitions from relative health through relative dysfunction or disease of the respective individual. The method further includes selecting one or more secondary stratification axes (e.g., sex, age, demographics, etc.) derived from data element(s) collected from the database of records of individuals. The method further includes training a model using data from individuals on whom the input data and the output data (e.g., ground truth output information) exist or can be imputed for each individual along the stratification axis or axes. The method further includes deploying a model to generate localized or probability distribution embeddings for each individual along the stratification axis or axes using input data associated with their health or information that correlates with their current or future health status. The method further includes stratifying each individual available to be recruited to the cross-sectional or longitudinal human study or trial based on a relationship of the respective collected data to the selected axis or axes. The method further includes determining a target distribution of patients to be recruited as a function of their position or probability distribution along the selected axes, where their position or probability along the selected axes is separate from the population distribution of the patients. The method further includes selecting and outputting a subpopulation of the plurality of individuals available to be recruited to the cross-sectional or longitudinal human study or trial whose collective distribution of positions or probabilities along the selected axis or axes is projected to resemble the target distribution.

In some embodiments, the method further includes training a model using data from individuals on whom input data and ground truth output information exist or can be imputed for each individual along the stratification axis or axes.

In some embodiments, the method further includes deploying a model to generate localized or probability distribution embeddings for each individual along the stratification axis or axes using input data associated with their health or information that correlates with their current or future health status.

In some embodiments, the method includes collecting data of a plurality of individuals.

In some embodiments, the data of the plurality of individuals includes: demographics, medical encounter, physical examination, diagnosis, medication, procedure, vital measurement, vaccination, representations of lab result(s), collection and/or analysis of serum, urine or other bio-sample, imaging, clinical note, radiology report, genetic test or report, biomarker, pathology report, other documented information associated with health, social determinants of health, financial status or behavior, or consumer status or behavior.

In some embodiments, the stratification axis or axes selected may be one or more primary stratification axes and/or may span from a state of relative health to a state of relative disease. In some embodiments, the stratification axis or axes may include one or more secondary stratification axes and/or may be selected based on factors such as a known or hypothesized relationship between the axis or axes and the disease state of interest or the availability of data elements corresponding to the axis or axes.

In some embodiments, the method further comprises, selecting a secondary stratification axis or axes that represent one or more properties about the plurality of individuals, including properties such as physiological comorbidities, demographics, or socioeconomic variables.

In some embodiments, the human study or trial is intended to study the progression of transitions in health, validate a biomarker, validate a panel of biomarkers, validate a target, or evaluate the efficacy or safety of a pharmacologic, digital, or other form of intervention, action or behavior modification in a population or subpopulation. In some embodiments, studying the progression of transitions in health can be performed by a fully-automated system, a semi-automated system, or is performed by a human after an automated metric is generated representing the progression.

In some embodiments, the selected subpopulation is enriched in one or more properties including: reduced heterogeneity in physiology, increased probability of outcome events, reduced heterogeneity in rate of progression, increased propensity to respond to an intervention, increased likelihood of observing specific physiological or pathological signs, or, more broadly, increased homogeneity in terms of expected endpoints.

In some embodiments, the stratification axis represents a continuous or ordinal dimension.

In some embodiments, the stratification axis or axes include a single data element or a combination of multiple data elements collected from a plurality of individuals.

In some embodiments, the position of individuals along a stratification axis or axes may be represented by either a point, point estimate or a probability distribution.

In some embodiments, the method further includes estimating, using imputation techniques, the position of individuals missing one or more relevant data elements wherein the imputation techniques can include multiple imputation or regression models.

In some embodiments, the stratification axis or axes includes learned parameter(s) derived from a model trained on data elements collected from the individuals.

In some embodiments, the determined target distribution is selected based on properties about the biology under evaluation, biomarker, panel of biomarkers, therapeutic target, or pharmacologic intervention to be evaluated.

In some embodiments, the target distribution is uniform with respect to the stratification axis or axes. In some embodiments, the method further includes discovering or validating a biomarker or panel of biomarkers. In some embodiments, the target distribution is enriched with respect to regions or subregions of one or more stratification axes. In some embodiments, the method further includes evaluating the efficacy of an intervention. In some embodiments, the selected target distribution may be substantially different from the background distribution of individual patients to recruit, such that an unguided recruitment protocol desiring to recruit the patients composing the target distribution would require many more recruits.

In some embodiments, the output subpopulation includes the individuals whose distribution along the stratification axis or axes statistically resemble the target distribution.

In some embodiments, the selected subpopulation enables rapid recruitment of individuals across a dimension which may otherwise require a longitudinal study. In some embodiments, the selected subpopulation enables efficient recruitment of individuals within a desired window of (e.g., portion of, subset of) the dimension.

In some embodiments, the selected subpopulation of individuals may be selected to minimize or maximize certain properties, such as the minimization of the total cost or number of patients required to maintain a minimum degree of coverage across the continuous dimension.

In some embodiments, the selected subpopulation of individuals may be selected to maintain certain distributions or balances of covariates, including demographics, covariates, comorbidities, medication usage, medical history, or other socioeconomic or biological properties.

In some embodiments, patients are selected by an optimization method, including linear programming, methods employing gradient descent, dynamic programming, or other schemes to approximate the identification of an optimal subset of recruits.

In some embodiments, the outputting the subpopulation may report an empty output subpopulation and that no subpopulation satisfying the target distributions and selection criteria exist. In some embodiments, in the event of an empty output subpopulation report, suggesting, to the trial designer, refinement(s) or relaxation(s) of certain target distributions or selection criteria that would return a non-empty output subpopulation.

In some embodiments, the method further includes outputting list(s) of patients who have comparable properties relative to the stratification axes, along with the number of patients to recruit from each list to satisfy the target distribution.

In some embodiments, outputting the subpopulation of individuals is performed using anonymized markers.

In some embodiments, some diseases have defined biomarkers indicating the disease or stage of the disease. In other diseases, such as liver disease, images can reveal how much fat or fibrosis is in the liver, and in Parkinson's disease the current standard of diagnosis is subjectively measurement of the individual's shaking or sleep disorders. Therefore, stratification can quantify an otherwise subjectively defined condition. The stratification can be in percentage groups, or ranges of a continuous score. The stratification can also be discrete groups.

In some embodiments, the stratification can be risk stratifications. In embodiments, the method can be employed to provide risk tiers for life insurance adjusters, or other risk-based applications.

In some embodiments, the method can provide clinical decision support, by providing a risk class derived from the probability distributions that are output by the model to a doctor, nurse, nurse practitioner, physician's assistant, or patient. In some embodiments, the doctor, nurse, nurse practitioner, physician's assistant, or patient can access the model over the Internet to determine diagnoses or staging. In some embodiments, the method can be provided to the doctor, nurse, nurse practitioner, physician's assistant, or patient as a Software-as-a-Service (SaaS) implementation. In some embodiments, the same probability distributions that are output by the model are used to provide clinical decision support and clinical trial enrichment simultaneously.

In some embodiments, the model is a machine-learning model or a neural network. The method can further employ supervised machine learning techniques that train on ordinal labels. In some embodiments, this predictive model is learned via ordinal regression, neural networks, generative models, random forests, nearest neighbors, support vector machines, and gaussian processes. A person of ordinary skill in the art that can understand that other statistical, machine learning methods, and neural networks can be employed.

In some embodiments, the transition in the health state is represented by a continuous dimension or a discrete dimension.

In some embodiments, the method further includes identifying or validating a biomarker or panel of biomarkers that indicates a transition in health state, biological function, physiological function, disease or a plurality of states. In some embodiments, the biomarker can be a paired biomarker that defines a pairing of intervention of biomarkers. The method can determine that two biomarkers may be paired in indicating a condition, or that a biomarker can be paired with an intervention. In some embodiments, the method further includes evaluating, using the model, whether a relationship exists between the biomarker or panel of biomarkers and the transition in health state or disease. The model is a machine learning model or a neural network. In some embodiments, the method further includes characterizing a relationship between the biomarker or panel of biomarkers and the transition in health state or disease. The relationship includes a probability that the biomarker or panel of biomarkers indicates the transition in health state, a confidence interval of the probability, and a range of a time to progression based on a range of values of the biomarker. In some embodiments, the biomarker or panel of biomarkers are validated in a human study whose participants are members of a model-identified subpopulation.

In some embodiments, the method further includes determining if an arbitrary intervention has an effect in a human study or trial with the model-selected subpopulation. In some embodiments, the arbitrary intervention can include a pharmaceutical intervention, a behavioral intervention, a digital intervention, a data collection, or other intentions or other actions. In some embodiments, a digital intervention can be a mobile application or computer application (e.g., “app”) that provides behavior modification, information about adherence, engagement with the application such as entering in health metrics, recording food or exercise data, etc. In some embodiments, the human study or trial includes determining which stages of health state, biological function, physiological function or disease does a particular intervention have an effect. In embodiments, the arbitrary intervention can be the method determining correlations with interventions in individual's existing medical records and biomarker changes.

In some embodiments, the method further includes enriching a human study or trial by selecting the subpopulation being at a specific stage of the health state, biological function, physiological function, or disease such that the impact of an intervention under test can be more precisely determined.

In some embodiments, the method further includes enriching a human study or trial by selecting a subpopulation based on a forecasted progression such that the impact of an intervention under test can be determined.

In some embodiments, the forecasted progression is a known or existing medical consensus stage.

In some embodiments, the method further includes conducting a human study or trial for evaluating the efficacy and safety of a protocol that uses and applies an intervention directed by a model for the subpopulation.

In some embodiments, the model is configured to identify individuals in the subpopulation that should receive an intervention, when the identified individuals should receive an intervention, or what intervention dose or regimen is used.

In some embodiments, outputting the subpopulation of individuals employs anonymized markers.

In some embodiments, the model is a machine-learning model or a neural network.

In some embodiments, the method further includes enrolling the subpopulation in a clinical trial based on the stratification. In some embodiments, the method further includes retraining the model based on the results of the clinical trial.

In some embodiments, the method further includes treating the subpopulation with a therapy based on the stratification. In some embodiments, the method further includes retraining the model based on the results of the clinical trial.

In some embodiments, a method of training a machine-learning model for running a cross-sectional or longitudinal human study or trial includes separating data from a database of records of a plurality of individuals into input data and output data. The input data is static or temporal features of a respective individual collected either passively or through active engagement. The output data is data that represents transitions from health through dysfunction or disease of the respective individual. The method includes defining one or more stratification axes derived from one or more data elements collected from the database of records of the plurality of individuals. The method further includes training a model using data from individuals on whom the input data and the output data (e.g., ground truth output information) exist or can be imputed for each individual along the stratification axis or axes, the trained model being configured to be deployed to: (i) generate localized or probability distribution embeddings for each individual along the stratification axis or axes using input data associated with their health or information that correlates with their current or future health status, (ii) stratify each individual of the plurality of individuals available to be recruited to the cross-sectional or longitudinal human study or trial based on a relationship of the respective collected data to the selected axis or axes, (iii) determine a target distribution of subjects to be recruited defined by the goals of the study or trial as a function of their position or probability distribution along the selected axes, their position or probability along the selected axes separate from the population distribution of the patients, and (iv) select and output a subpopulation of the plurality of individuals available to be recruited to the cross-sectional or longitudinal human study or trial whose collective distribution of positions or probabilities along the selected axis or axes is projected to resemble the target distribution.

In some embodiments, a method of running a model trained to select a population for a cross-sectional or longitudinal human study or trial includes deploying a model to generate localized or probability distribution embeddings for each individual along the stratification axis or axes using input data associated with their health or information that correlates with their current or future health status. The model is trained on a database of records of individuals separated into input data and output data. The input data is/are static or temporal features of a respective individual collected either passively or through active engagement. The output data is/are data that represents transitions from health through dysfunction or disease of the respective individual. Stratification axes can be defined based on one or more data elements collected from the database of records of the plurality of individuals. The model is trained using data from individuals on whom the input data and the output data (e.g., ground truth output information) exist or can be imputed for each individual along the stratification axis or axes. Stratifying each individual of the plurality of individuals available to be recruited to the cross-sectional or longitudinal human study or trial based on a relationship of the respective collected data to the selected axis or axes. The method further includes determining a target distribution of subjects to be recruited defined by the goals of the study or trial as a function of their position or probability distribution along the selected axes, their position or probability along the selected axes separate from the population distribution of the patients. The method further includes selecting and outputting a subpopulation of the plurality of individuals available to be recruited to the cross-sectional or longitudinal human study or trial whose collective distribution of positions or probabilities along the selected axis or axes is projected to resemble the target distribution.

### Terminology

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

Measurements, sizes, amounts, etc. may be presented herein in a range format. The description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as 10-20 inches should be considered to have specifically disclosed subranges such as 10-11 inches, 10-12 inches, 10-13 inches, 10-14 inches, 11-12 inches, 11-13 inches, etc.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

While example embodiments have been particularly shown and described, it will be understood by those skilled in the art that various changes in form and details may be made therein without departing from the scope of the embodiments encompassed by the appended claims.

1. A method of performing a clinical trial or biomarker validation study, the method comprising:
obtaining access to a computer-implemented model that was trained using training data comprising a plurality of records for a plurality of individuals, each record comprising (i) values for a plurality of features for a respective individual from the plurality of individuals and (ii) a label providing an indication of a disease state for the respective individual;
providing, to the trained computer-implemented model, a plurality of sets of values for the plurality of features, each set of values corresponding to a candidate individual from a set of candidate individuals;
receiving, from the trained computer-implemented model, a prediction of a disease state for each set of values;
identifying, from the predictions of the disease state, a group of participants from the set of candidate individuals; and
facilitating at least one of a clinical trial or a biomarker validation study involving the group of participants.

2. The method of claim 1, wherein the values for the plurality of features describe at least one of a demographic, a medical encounter, a physical examination, a diagnosis, a medication, a medical procedure, a vital measurement, a vaccination, a lab result, a serum, a urine sample, a bio-sample, a gene expression level, a medical image, a clinical note, a radiology report, a genetic test, a biomarker, a pathology report, health information, a social determinant of health, financial data, consumer data, or any combination thereof.

3. The method of claim 1, wherein the model comprises at least one of a regression model, a classifier, a linear model, a non-linear model, a random forest, a kernel method, a Bayesian model, a decision tree, or a neural network.

4. The method of claim 1, wherein the prediction of the disease state comprises a probability distribution.

5. The method of claim 1, wherein the prediction of the disease state is on a primary stratification axis comprising a health-to-disease axis or a disease risk axis.

6. The method of claim 1, wherein identifying the group of participants comprises stratifying each candidate individual from the plurality of candidate individuals according to one or more primary stratification axes.

7. The method of claim 6, wherein identifying the group of participants further comprises stratifying each candidate individual from the plurality of candidate individuals according to one or more secondary stratification axes, and wherein the one or more secondary stratification axes represent one or more properties of the plurality of individuals, the properties comprising at least one of a physiological comorbidity, a demographic, or a socioeconomic variable.

8. The method of claim 7, wherein at least one stratification axis from the one or more primary stratification axes or the one or more secondary stratification axes represents at least one of a continuous dimension or an ordinal dimension.

9. The method of claim 7, wherein a position along at least one stratification axis from the one or more primary stratification axes or the one or more secondary stratification axes is represented by a point, a point estimate, or a probability distribution.

10. The method of claim 1, wherein the clinical trial or the biomarker validation study comprises studying at least one of a progression of transitions in health, validity of a biomarker, validity of a panel of biomarkers, validity of a target therapeutic, efficacy of a therapeutic intervention, efficacy of a digital intervention, efficacy of a behavioral intervention, or any combination thereof.

11. The method of claim 1, wherein the group of participants is enriched in one or more properties relative to the set of candidate individuals, the one or more properties comprising at least one of reduced heterogeneity in physiology, increased probability of outcome events, increased propensity to respond to an intervention, increased likelihood of observing specific physiological or pathological signs, or any combination thereof.

12. The method of claim 1, wherein identifying the group of participants comprises:
determining a target distribution for the set of candidate individuals according to a goal of the clinical trial or the biomarker validation study, the target distribution defining a probability distribution; and
selecting the group of participants from the set of candidate individuals to achieve a collective distribution for the group of participants that resembles the target distribution.

13. The method of claim 12, wherein the target distribution is determined based on at least one parameter to be evaluated in the clinical trial or the biomarker validation study, the at least one parameter comprising at least one of a biological property, a biomarker, a panel of biomarkers, a therapeutic target, or a pharmacologic intervention.

14. The method of claim 12, wherein the target distribution is uniform with respect to the disease state.

15. The method of claim 12, wherein the target distribution comprises a risk probability distribution.

16. The method of claim 12, wherein the target distribution is enriched with respect to a region of a stratification axis associated with the disease state.

17. The method of claim 12, wherein selecting the group of participants comprises minimizing a number of individuals in the group of participants.

18. The method of claim 12, wherein selecting the group of participants comprises:
identifying multiple groups of individuals, wherein each group of individuals from the multiple groups of individuals has a similar probability distribution with respect to the disease state; and
determining a number of individuals from each group of individuals to include in the group of participants to satisfy the target distribution.

19. The method of claim 1, wherein the method comprises facilitating the clinical trial, and wherein the clinical trial comprises evaluating the efficacy of a treatment.

20. The method of claim 1, wherein the method comprises facilitating the clinical trial, and wherein facilitating the clinical trial comprises registering members from the group of participants to the clinical trial, such that the members are administered a medication, a digital intervention, or a behavioral intervention.

21. The method of claim 1, wherein the method comprises facilitating the biomarker validation study, and wherein the biomarker validation study comprises validating a biomarker or panel of biomarkers.

22. The method of claim 1, wherein identifying the group of participants comprises achieving a desired distribution of at least one covariate, the at least one covariate comprising at least one of a demographic, a comorbidity, a medication usage, a medical history, a socioeconomic property, or a biological property.

23. The method of claim 1, further comprising training the computer-implemented model using the training data.

24. A system for performing a clinical trial or biomarker validation study, the system comprising one or more computer processors programmed to perform operations comprising:
obtaining access to a computer-implemented model that was trained using training data comprising a plurality of records for a plurality of individuals, each record comprising (i) values for a plurality of features for a respective individual from the plurality of individuals and (ii) a label providing an indication of a disease state for the respective individual;
providing, to the trained computer-implemented model, a plurality of sets of values for the plurality of features, each set of values corresponding to a candidate individual from a set of candidate individuals;
receiving, from the trained computer-implemented model, a prediction of a disease state for each set of values;
identifying, from the predictions of the disease state, a group of participants from the set of candidate individuals; and
facilitating at least one of a clinical trial or a biomarker validation study involving the group of participants.

25. The system of claim 24, wherein the prediction of the disease state is on a primary stratification axis comprising a health-to-disease axis or a disease risk axis.

26. The system of claim 24, wherein identifying the group of participants comprises stratifying each candidate individual from the plurality of candidate individuals according to one or more primary stratification axes and one or more secondary stratification axes.

27. The system of claim 24, wherein identifying the group of participants comprises:
determining a target distribution for the set of candidate individuals according to a goal of the clinical trial or the biomarker validation study, the target distribution defining a probability distribution; and
selecting the group of participants from the set of candidate individuals to achieve a collective distribution for the group of participants that resembles the target distribution.

28. The system of claim 27, wherein the target distribution comprises a risk probability distribution.

29. The system of claim 27, wherein selecting the group of participants comprises:
identifying multiple groups of individuals, wherein each group of individuals from the multiple groups of individuals has a similar probability distribution with respect to the disease state; and
determining a number of individuals from each group of individuals to include in the group of participants to satisfy the target distribution.

30. The system of claim 24, the operations further comprising training the computer-implemented model using the training data.
